(19) United States
(12) Patent Application Publication
(10) Pub. No.: US 2009/0270928 A1
(43) Pub. Date: Oct. 29, 2009

(54) NAVIGATION TRACKER FIXATION DEVICE AND METHOD FOR USE THEREOF

(76) Inventors: Ross G. Stone, Boca Raton, FL (US); Logan D. Stone, Boca Raton, FL (US)

Correspondence Address: OSTROLENK FABER GERB & SOFFEN, 1180 AVENUE OF THE AMERICAS, NEW YORK, NY, 100368403, US

(21) Appl. No.: 12/430,461

(22) Filed: Apr. 27, 2009

Related U.S. Application Data
(60) Provisional application No. 61/047,974, filed on Apr. 25, 2008.

(51) Int. Cl. A61B 17/80; A61B 5/05

(52) U.S. Cl. 606/286; 600/424

(57) ABSTRACT

A navigation tracker fixation device system is provided that comprises a plate that is placed at a generally longitudinal axis of a patient's bone and having at least one hole and operable to receive a member for coupling the plate to the bone. A post is provided that is coupled to the plate and operable to extend beyond a surgical site. A tracker component may be coupled to the plate, the post or both the plate and the post, and the post enables the tracker component to be positioned within the surgical site while remaining away from an articular surface resection area of the bone. The tracker component remains within the surgical site during resection of the bone. Preferably, the surgical tracking system is provided to verify bone resection or knee kinematics, without a need to remove or replace the tracker component from the surgical site, during the procedure.

### CROSS-REFERENCE TO RELATED APPLICATION

This application is related to and claims priority to U.S. provisional patent application Ser. No. 61/047,974, entitled KNEE REPLACEMENT NAVIGATION TRACKER FIXATION DEVICE AND METHOD FOR USE THEREOF and filed on Apr. 25, 2008, the entire contents of which is incorporated by reference herein in its entirety.

### BACKGROUND

1. Field of the Invention

The invention relates, generally, to surgical devices, and, more particularly, to an improved surgical navigation tracking system and method.

2. Description of the Related Art

In recent years, only approximately three percent of roughly 300,000 knee replacement surgeries in the United States were performed using computer-assisted navigation. Computer-assisted navigation for surgeons include technology to display anatomical images on a monitor, and further to display surgical instruments orientation, vis-à-vis skeletal tissues, used during a procedure. Over the past ten years, computer navigation has radically changed brain neurosurgical procedures, such as developed by corporations, including BRAINLAB, NAVITRACK, ASCULAP, MEDTRONIC and STRYKER. Recently, computer-assisted navigation is used in orthopedic procedures, such as joint replacement, as well.

Computer-assisted orthopedic surgery utilizes techniques for providing visibility of surgical anatomy, as well as to provide quantitative feedback for surgeons that increase accuracy in a procedure. Such feedback is particularly important in orthopedic surgical procedures, where positioning of prosthetics or devices requires accuracy of, for example, up to one millimeter or less, and/or three degrees or less. Any movement or placement of a prosthetic or device beyond a minimum amount can result in a failed surgical procedure. Accordingly, three-dimensional imaging or motion analysis provides important feedback and information before, during and after a surgical procedure.

In known orthopedic computer-assisted navigation systems, several (e.g., four to six) stab wounds or small incisions placed away from a main surgical incision area are required for placement of a tibial (leg bone) tracker and a femoral (thigh bone) tracker, which can cause additional bleeding and bone, skin and muscle damage away from the primary operative incision.

Prior art fixation systems for stab wound tracker bone fixation include two parallel pins or two parallel pins with a third pin placed not parallel to the other two which is used in the articular and stab wound techniques provided by STRYKER. Current tracker holding systems engage the bone pins well above the bone by grabbing two and three pins provided in configurations above the skin, subcutaneous tissue and muscle.

Examples of known optical tracking systems include active optical tracking systems and passive optical tracking systems. Active optical tracking systems typically include infrared light-emitting diodes (“LEDs”) that are viewable by cameras. The LEDs are, typically, rigidly attached to a tracker, and the tracker is fixed to the patient or an instrument. The cameras detect the light from the LEDs and calculate the position of the tracker and/or the instrument on the patient. Passive optical trackers, in contrast, include printed patterns or reflective markers that are detected by cameras that are provided with LED around the lens of the camera. The light that is emitted by the LEDs is reflected back to the camera(s) and the position of the tracker and/or the instrument on the patient is calculated.

One known system includes an articular surface method of tracker placement, which eliminates the above-identified problem of additional stab wounds, but has a disadvantage of early tracker removal prior to completion of a procedure. Since articular surface method trackers are positioned on the articular surface, which is resected, the trackers must be removed prior to resection and then replaced on the resected or cut surface of the femur with renavigation of the femoral head to validate the distal femoral resection (bone cut), which adds time to the operative procedure. Accordingly, the trial resection cannot be validated or verified during the procedure using the articular surface method.

### SUMMARY

Unlike known prior art systems, the replacement navigation tracker fixation device system disclosed herein precludes a need for additional stab wounds, as well as precludes a need to remove a tracker during a surgical procedure. Preferably, a tracker in accordance with the teachings herein lays flat on exposed bone. In a preferred embodiment, a surgical tracking system is provided that comprises a plate and operable to receive a member for coupling the plate to a flat or slightly curved surface of a patient's bone. Further, a post is provided that is coupled to the plate and operable to extend beyond a surgical site. Moreover, a tracker component is coupled to the plate or to the post, wherein the post enables the tracker component to be positioned within the surgical site while remaining away from an articular surface resection area of the bone, and further wherein the tracker component remains within the surgical site during resection of the bone. Alternatively, the tracker is coupled to both the plate and the post. Preferably, the surgical tracking system is provided to verify bone resection without a need to remove or replace the tracker component from the surgical site. Also, knee joint kinematics may be demonstrated during a tissue reduction or after femoral implantation.

In a preferred embodiment, the plate is fixed to a patient's femur, and a second plate is provided that has at least one hole and is operable to receive a member for coupling the plate to a flat surface of the patient's tibia. A second post is preferably coupled to the second plate and extends beyond the surgical site. A second tracker component is fixed to a generally longitudinal axis of the tibia, wherein the second post enables the second tracker component to be positioned within the surgical site while remaining away from an articular surface resection area of the tibia, and further wherein the tracker component remains within the surgical site during a resection of the tibia.

Other features and advantages of the present invention will become apparent from the following description of the invention that refers to the accompanying drawings.

### BRIEF DESCRIPTION OF THE DRAWINGS

For the purpose of illustrating the invention, there is shown in the drawings a form which is presently preferred, it being understood, however, that the invention is not limited to the precise arrangements and instrumentalities shown. The features and advantages of the present invention will become apparent from the following description of the invention that refers to the accompanying drawings, in which:

FIG. 1 is an anatomical drawing illustrating a patient's knee and showing dotted lines indicating placement for an incision of a surgical procedure using the knee replacement navigation tracker fixation device in accordance with the teachings herein;

FIG. 2 illustrates a step in the procedure for using the knee replacement navigation tracking fixation device in accordance with a preferred embodiment;

FIG. 3 illustrates placement of the knee replacement navigation tracker fixation system in accordance with an embodiment;

FIG. 4 illustrates an example placement of a plate in accordance with an embodiment;

FIG. 5 illustrates an example placement of a plate in accordance with another embodiment;

FIGS. 6A and 6B illustrate example screws that are preferably used to fix a plate to a patient's bone in accordance with an example embodiment;

FIGS. 7A-7H illustrate example embodiments and configurations of plates and/or posts to be fixed to various bones at various locations and in various ways;

FIG. 8 is an anatomical diagram illustrating a top view knee replacement navigation tracker fixation system during a surgical procedure in accordance with an embodiment;

FIG. 9 is another anatomical diagram illustrating a top view knee replacement navigation tracker fixation system during a surgical procedure in accordance with an embodiment; and

FIG. 10 is another anatomical diagram illustrating the coupling of plates and trackers to a patient's femur and tibia, respectively, in accordance with an embodiment.

### DESCRIPTION OF THE EMBODIMENTS

In a preferred embodiment, a fixation system is provided that includes a small plate that is coupled with two holes, one hole on each side of a standing post. The plate is preferably configured in a rectangular, triangular, square or hexagonal shape to accept one or more screws in the optimal geometrical configuration for the number of screws to stabilize the post. The holes in the plate preferably receive screws that fix the plate to bone. In one embodiment, the holes of the plate are threaded. The post may be provided in various forms, including a triangular, hexagonal or other shaped rod. The post is preferably rigidly fixed to the plate, and provides medial tissue retraction. In one embodiment, a blade is coupled to the post for the medial tissue retraction. In another embodiment, a support is preferably coupled to the post and operable to support a cutting jig for bone resection.

When the fixation system is surgically installed, the post preferably extends out of the wound. Further, the fixation system according to the teachings herein, particularly the post, preferably includes one or more curved portions that allow one or more tracker components to reside along the general longitudinal axis of the tibia or femur without interfering with a surgeon's hands during a procedure. The tracker component(s) are placed away from the wound site, preferably due to the curvature of the post. Moreover, the tracker components are preferably fixed and secured such that it is compatible with all existing tracker systems. In a preferred embodiment, the plate is fixed to the bone on a flat surface, and secured with two standard cancellous (spongiosa) screws in the metaphyseal area of the tibia and/or femur on the medial side thereof.

By placing the femoral and tibial trackers inside the wound, a surgeon can validate and verify bone resections, as well as knee kinematics (knee alignment during range of motion), with the trial and final replacement implants, which achieves the same information obtained by trackers positioned outside the wound, without the extra stab wounds which cause skin scars, additional blood loss and muscle damage.

In a preferred embodiment, a knee replacement navigation tracker device is fixed to the bone from inside a surgical site. Unlike prior art navigation tracker devices, however, the device is preferably placed or fixed away from the operative articular surface resection area for implant placement during the surgical procedure.

In a preferred embodiment, the tibia fixation area is slightly over ten millimeters below the articular surface and above the distal end of the tibial tubercle on the medial side of the tibia, which is exposed during surgery by subperiosteal dissection. The femoral fixation area is preferably on the medial side of the femur, slightly over ten millimeters above the distal end of femoral articular surface, and approximately midway between the anterior and posterior margins of the femoral surface. In alternative embodiments, various rigid plate and screw configurations are provided, such as a triangular plate with three screws at the corners, a square plate with four screws or a round plate with multiple screws. Moreover, the screws may be simple threaded screws, or may be locking screws with threads placed just below the screw head to engage threaded plate holes, and additional, primary threads of a different pitch and size to hold in the bone. In one embodiment, the plate is a locking plate and provided with screw holes that are threaded to engage threads in locking screws that are located just below the screwheads. Preferably, the locking screw threads are separate and distinct from the screw threads that are positioned lower on the screw shaft and that physically engage the patient's bone. The combination of a locking plate with locking screws forms a more robust construction than a non-locking plate and screw construction.

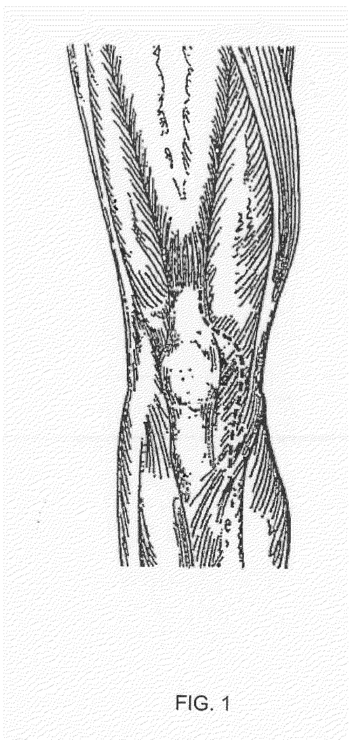

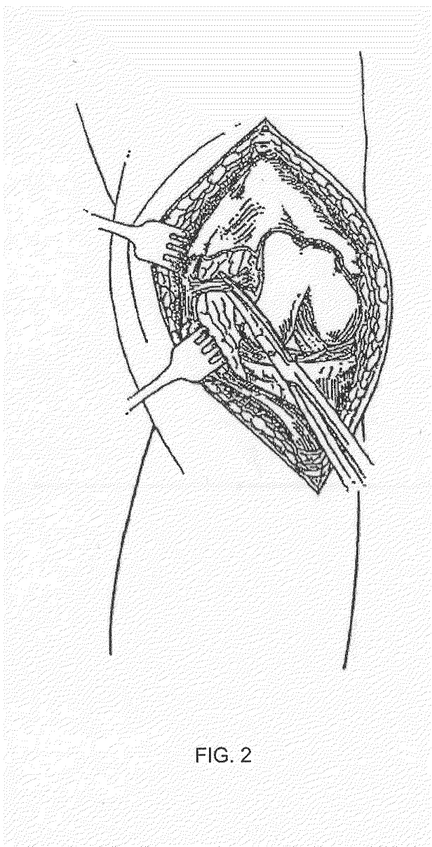

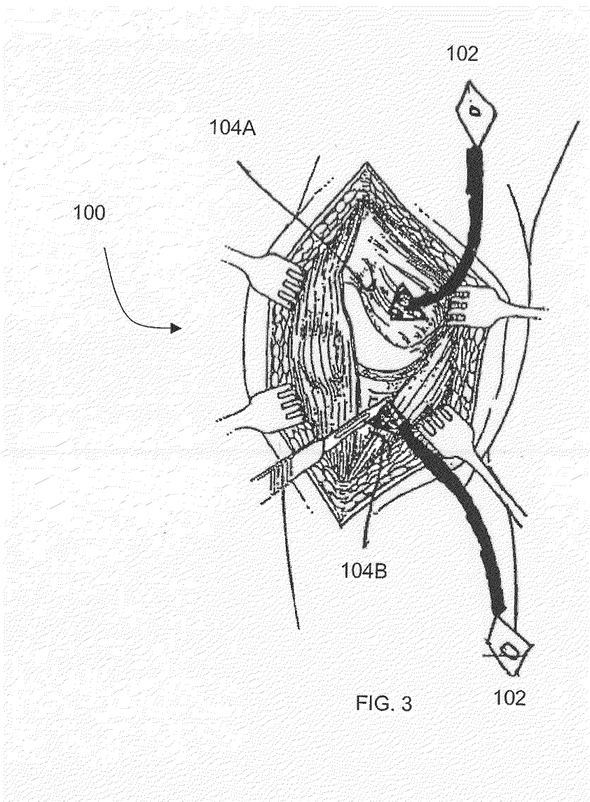

Referring to the drawings, in which like reference numerals represent like elements, FIG. 1 is an anatomical drawing illustrating a patient's knee and showing dotted lines indicating placement for an incision of a surgical procedure using the knee replacement navigation tracker fixation device in accordance with the teachings herein. More particularly, the incision is appropriate for a medial parapetellar retinacular approach. FIG. 2 illustrates the procedure, including that lateral patellofemoral plicate are cut to allow mobilization of extensor mechanism. FIG. 3 illustrates placement of navigation tracker fixation system 100 in accordance with a preferred embodiment. During the surgical procedure, the medial capsule and a deep portion of MCL are elevated subperiorsteally. As shown in FIG. 3, navigation tracker fixation system 100 includes tracker component 102. Tracker component 102 may be any appropriate tracker element, as known in the art. Also shown in FIG. 3, navigation tracker fixation system 100 includes plates 104A and 104B, which are preferably fixed to the patient's tibia and femur, respectively, on a flat surface, and secured with two screws (not shown). In a preferred embodiment, plate 104 operates as a locking plate, by locking to the patient's bone and integrated with tracker component 102.

FIG. 4 illustrates an example placement of plate 104 in accordance with an embodiment. In the embodiment shown in FIG. 4, plate 104 is a femoral locking plate and fixed to a flat surface of the medial side of the femoral condyle.

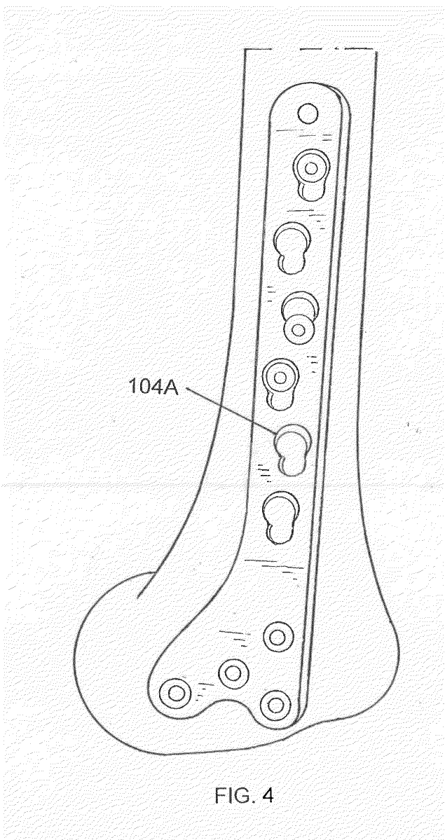

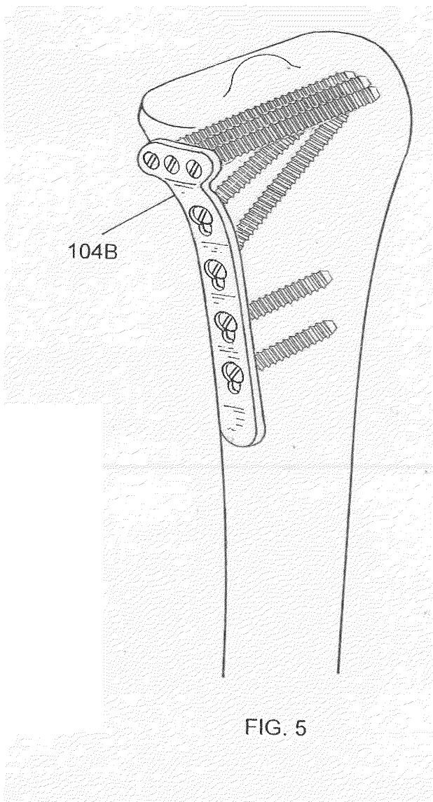

FIG. 5 illustrates an example placement of plate 104 in accordance with an embodiment. In the embodiment shown in FIG. 5, plate 104 is a tibial locking plate and fixed to the medial metaphysis of the tibia.

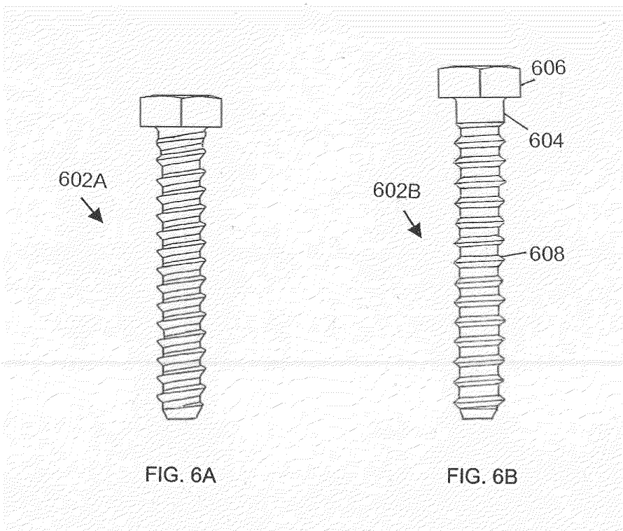

FIGS. 6A and 6B illustrate example screws 602A that are preferably used to fix plate 104 to a patient's bone. In the example shown in FIG. 6A, screw 602A is a standard threaded screw and has a first pitch and size for plate 104 the bone. In the example shown in FIG. 6B, screw 602B is a locking screw having threads 604 that are located adjacent to or substantially adjacent to screw head 606. Threads 604 are preferably provided for the threads to engage a threaded hole in plate 104. In addition to threads 604, primary threads 608 are preferably provided that enable screw 602B to hold in the bone. In a preferred embodiment, threads 608 are of a different pitch and size than threads 604. The difference in size and pitch between threads 604 and 608 enables screw 602B to engage holding plate 104 and a patient's bone, respectively.

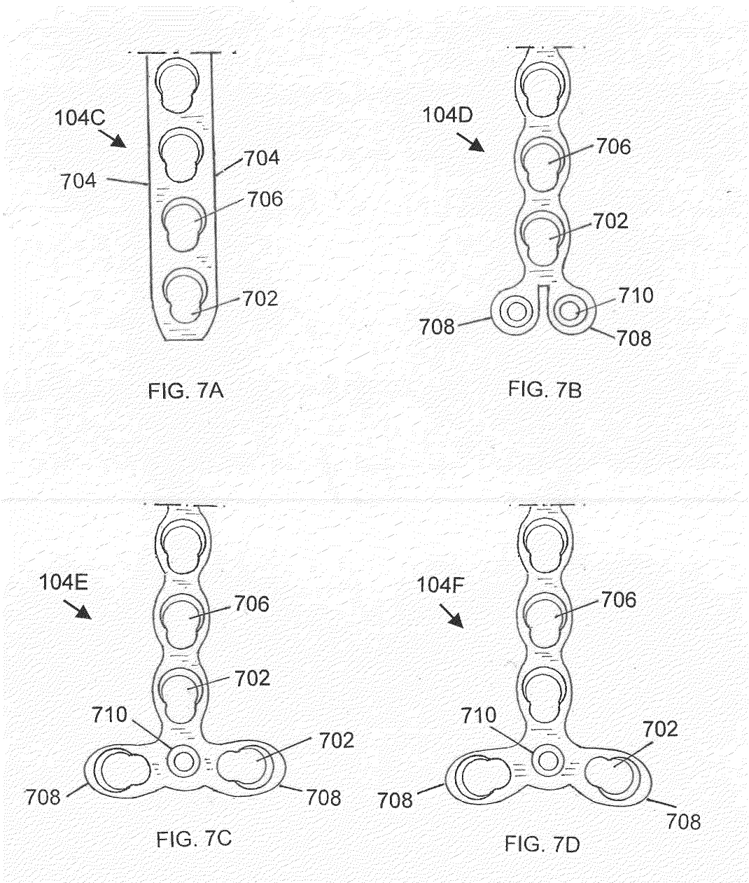

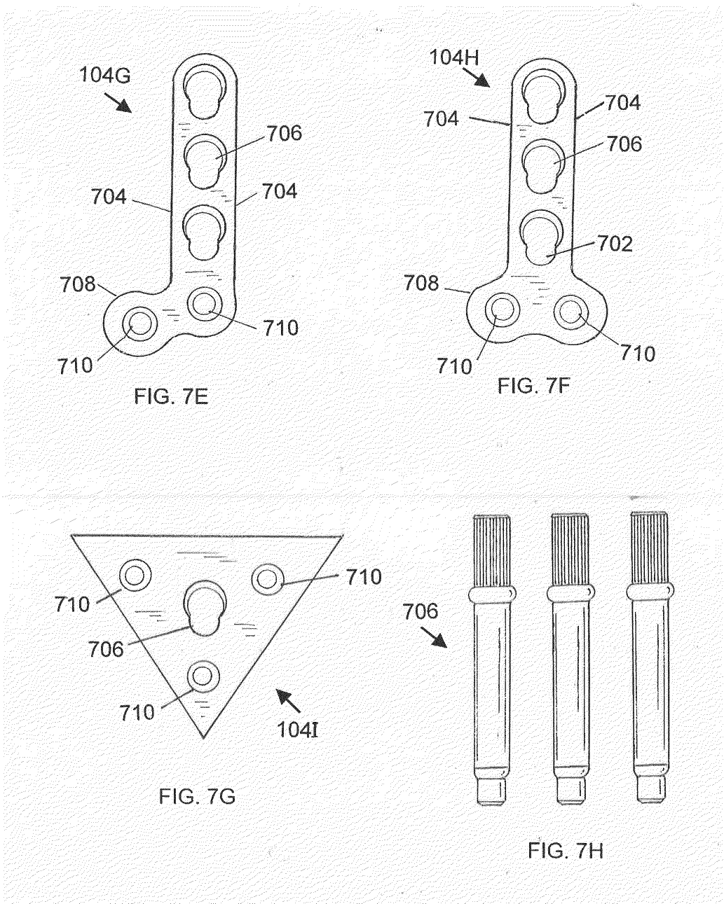

FIGS. 7A-7G illustrate example embodiments of plates 104C-104I, respectively, that represent various plate 104 configurations and useful for fixing plate 104 to various bones at various locations and in various ways. As shown in FIGS. 7A-7G, plates 104 include apertures 704 that receive screws 602. In one embodiment, apertures 104 are threaded and enable screw 602 to be threaded therein.

In the embodiment shown in FIG. 7A, plate 104C is substantially straight and includes no cantilever portions. Apertures 702 are provided that may be threaded to receive screw 602 for fixing plate 104C to bone. In the embodiment shown in FIG. 7A, plate 104C includes sidewalls 704 that are relatively wide, each sidewall 704 being approximately 25% of the total width of plate 104C. The design of plate 104C provides for a relatively strong construction and is inexpensive to manufacture. Due to its simple design, plate 104C has limited use for portions of straight bone. Plate 104C is preferably provided with post 706 (not shown), which may be positioned along the longitudinal axis of plate 104C, depending upon the application and use of plate 104C at one or more of apertures 702.

In the embodiment shown in FIG. 7B, plate 104D is substantially straight and includes two cantilever portions 708. In the embodiment shown in FIG. 7B, cantilever portions 708 extend from plate 104D at an angle of approximately 150°. Apertures 710 are provided within cantilever portions 708 and may be threaded to receive screw 602 for fixing plate 104D to bone, and are preferably circular. Plate 104D is preferably provided with post 706 (not shown), which may be positioned along the longitudinal axis of plate 104D, depending upon the application and use of plate 104D, at one or more of apertures 702. In the embodiment shown in FIG. 7B, plate 104D does not include sidewalls. Due to its design, plate 104D is fixed to the bone, such as above the distal end of the tibial tubercle on the medial side of the tibia via cantilever portion 708.

FIG. 7C illustrates an example plate 104E, which is similar to the example embodiment shown in FIG. 7B, and also is substantially straight and includes two cantilever portions 708. In the embodiment shown in FIG. 7C, cantilever portions 708 extend from plate 104E at an angle of approximately 90°. In the example shown in FIG. 7C, aperture 710 is provided between cantilever portions 708 that may be threaded to receive screw 602 for fixing plate 104E to bone, and is preferably circular. In the embodiment shown in FIG. 7C, plate 104E does not include sidewalls. Due to its design, plate 104E is fixed to the bone, such as above the distal end of the tibial tubercle on the medial side of the tibia, via cantilever portions 708.

FIG. 7D illustrates an example plate 104F, which is similar to the example embodiments shown in FIGS. 7B and 7C, and also is substantially straight and includes two cantilever portions 708. In the embodiment shown in FIG. 7D, cantilever portions 708 extend from plate 104F at an angle of approximately 120°. In the example shown in FIG. 7C, aperture 710 is provided between cantilever portions 708 that may be threaded to receive screw 602 for fixing plate 104F to bone, and is preferably circular. In the embodiment shown in FIG. 7D, plate 104F does not include sidewalls. Due to its design, plate 104F is fixed to the bone, such as above the distal end of the tibial tubercle on the medial side of the tibia, via cantilever portions 708.

FIG. 7E illustrates an example plate 104G, which, unlike the example embodiments shown in FIGS. 7B, 7C and 7D, includes a single cantilever portion 708. In the embodiment shown in FIG. 7E, plate 104G includes sidewalls 704 that are relatively wide, each sidewall 704 being approximately 25% of the total width of plate 104G. In the embodiment shown in FIG. 7E, cantilever portion 708 extends from plate 104G at an angle of approximately 110°. In the example shown in FIG. 7E, aperture 710 is provided at the proximal end of cantilever portion 708 that may be threaded to receive screw 602 for fixing plate 104G to bone, and is preferably circular. Due to its design, plate 104G is fixed to the bone, such as above the distal end of the tibial tubercle on the medial side of the tibia, via cantilever portion 708.

FIG. 7F illustrates an example plate 104H, which is similar to the example embodiments shown in FIGS. 7B and 7C, and also is substantially straight and includes two cantilever portions 708. In the embodiment shown in FIG. 7F, cantilever portions 708 extend from plate 104H at an angle of approximately 125°, and are beveled. In the embodiment shown in FIG. 7F, plate 104H includes sidewalls 704 that are relatively wide, each side wall 704 being approximately 20% of the total width of plate 104H. In the example shown in FIG. 7F, aperture 710 is provided between cantilever portions 708 that may be threaded to receive screw 602 for fixing plate 104H to bone, and is preferably circular. Due to its design, plate 104H is fixed to the bone, such as above the distal end of the tibial tubercle on the medial side of the tibia, via cantilever portions 708.

FIG. 7G illustrates an example plate 7041 in accordance with a preferred embodiment. In the embodiment shown in FIG. 7G, plate 104I is substantially triangular, and includes no cantilever portions that extend from plate 104I. Apertures 710 are preferably provided with plate 104I that may be threaded to receive screw 602 for fixing plate 104I to bone, and are preferably circular. In the embodiment shown in FIG. 7G, plate 104I does not include sidewalls. Preferably, curved post 706 is provided at or near the center of plate 104I and lies along the medial skin of the tibia and femur, respectively.

FIG. 7H illustrates example posts 706 that are rigidly coupled to plate 104 via aperture 702. During a surgical procedure, post 706 preferably extends out of the wound.

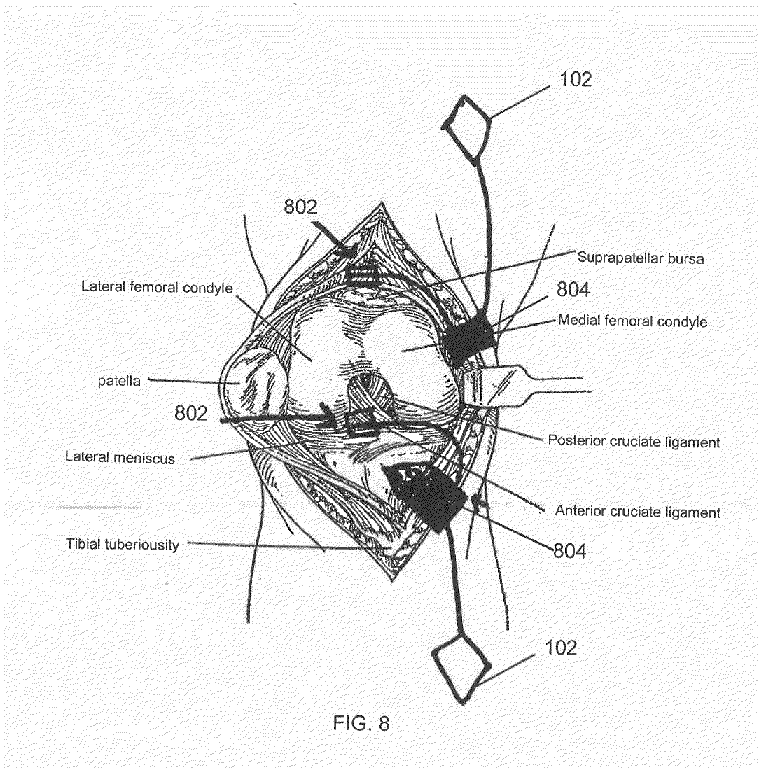

FIG. 8 is an anatomical diagram illustrating a top view of navigation tracker fixation system 100 during a surgical procedure, such as a knee replacement surgery. As shown in FIG. 8, two tracker components 102 reside along the general longitudinal axis of the tibia or femur, respectively. Cutting jig supports 802 are preferably provided at the suprapatellar bursa, and the lateral meniscus and anterior cruciate ligament, respectively. Further, retractor blades 804 provide tissue retraction, and enable tracker components 102 to remain placed during resection, thereby enabling verification of the resection while the bone is being resected. As noted above, cutting jig supports are coupled to post 706 (not shown) for bone resection.

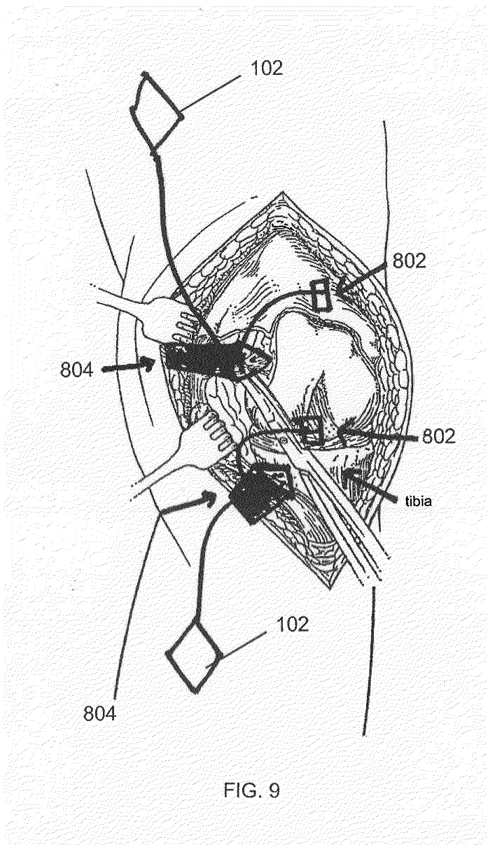

FIG. 9 illustrates another anatomical diagram of a top view knee replacement navigation tracker fixation system during a surgical procedure in accordance with an embodiment. As shown in FIG. 9, two tracker components 102 reside along the general longitudinal axis of the tibia or femur, respectively. Cutting jig supports 802 are preferably provided, and retractor blades 804 provide tissue retraction thereby enabling tracker components 102 to remain placed during resection, and enabling verification of the resection while the bone is being resected.

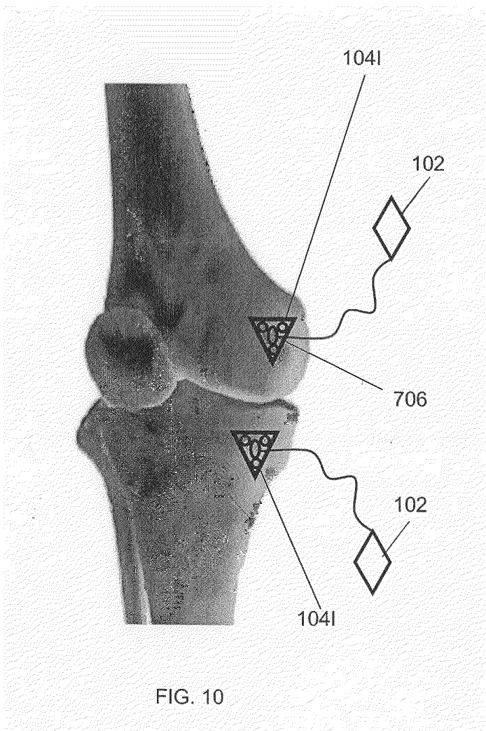

FIG. 10 is another anatomical diagram illustrating the coupling of plates 104 and tracker components 102 to a patient's femur and tibia, respectively, in accordance with an embodiment. As shown in FIG. 10, plate 104I is triangular, and includes post 706 to which tracker component 102 is coupled.

Thus, and in accordance with the teachings herein, navigation tracker fixation system 100 greatly improves computer navigation surgical procedures, such as for knee replacements. For example, several (e.g., four to six) stab wounds or small incisions placed away from a main surgical incision area that were required in the prior art for placement of the tibial (leg bone) tracker and femoral (thigh bone) trackers and that cause additional bleeding and muscle damage away from the primary operative incision are eliminated in accordance with the teachings herein. Further, navigation tracker fixation system 100 does not require early removal prior to completion of a surgical procedure, unlike in the prior art, and, accordingly, provide current and less invasive bone resection verification.

Although the present invention has been described in relation to particular embodiments thereof, many other variations and modifications and other uses will become apparent to those skilled in the art. For example, although many of the examples described herein relate to knee replacement surgery, other modifications and uses are envisioned, such as for hip replacement surgery, elbow replacement surgery or other surgeries requiring bone resection.

It is preferred, therefore, that the present invention be limited not by the specific disclosure herein.

What is claimed is:

1. A surgical tracking system, the system comprising:
a plate for receiving at least one member for coupling the plate to a generally longitudinal axis of a flat surface of a patient's bone;
a post coupled to the plate and operable to extend beyond a surgical site; and
a tracker component for navigation and coupled to the plate or coupled to the post, wherein the post enables the tracker component to be positioned within the surgical site while remaining away from an articular surface resection area of the bone, and the tracker component remains within the surgical site during resection of the bone.

2. The surgical tracking system of claim 1, wherein the resection is verified without removal or replacement of the tracker component.

3. The surgical tracking system of claim 1, wherein the plate is substantially triangular-shaped, square-shaped, hexagonal-shaped or rectangular-shaped.

4. The surgical tracking system of claim 1, wherein the bone is a femur, and further comprising a second plate operable to receive a member for coupling the plate to a flat surface of the patient's tibia;
a second post coupled to the second plate and operable to extend beyond the surgical site;
a second tracker component coupled to the second plate or to the second post, and securedly fixed to a generally longitudinal axis of the tibia, wherein the second post enables the second tracker component to be positioned within the surgical site while remaining away from an articular surface resection area of the tibia, and the second tracker component remains within the surgical site during resection of the tibia.

5. The surgical tracking system of claim 4, wherein the tibia resection and the femur resection are verified without removal or replacement of the tracker component and the second tracker component.

6. The surgical tracking system of claim 4, wherein the second tracker component is coupled to both the second plate and the second post.

7. The surgical tracking system of claim 1, wherein the post is a triangular, hexagonal, round or square shaped.

8. The surgical tracking system of claim 1, wherein the post or the plate has at least a curved portion, or both the post and the plate have at least a curved portion.

9. The surgical tracking system of claim 1, wherein the at least one member is one or more cancellous screws.

10. The surgical tracking system of claim 1, wherein at least one of the at least one member is a locking screw, and the plate includes at least one locking thread for receiving the at least one locking screw.

11. The surgical tracking system of claim 1, wherein the tracker component further verifies knee kinematics.

12. The surgical tracking system of claim 1, wherein the plate is placed in the metaphyseal area of the tibia or the medial side of the femur.

13. The surgical tracking system of claim 1, wherein the plate is fixed to the tibia at approximately ten millimeters below the proximal articular surface on the medial side of the tibia.

14. The surgical tracking system of claim 1, wherein the plate is fixed on the medial side of the femur over ten millimeters above the distal end of femoral articular surface.

15. The surgical tracking system of claim 1, further comprising at least one support coupleable to the post and operable to support a cutting jig for the bone resection.

16. The surgical tracking system of claim 1, wherein the tracker component is either a passive optical tracker or an active optical tracker.

17. The surgical tracking system of claim 1, further comprising at least one blade coupleable to the post and operable for medial tissue retraction at the surgical site.

18. The surgical tracking system of claim 1, wherein the tracker component is coupled to both the plate and the post.

19. A method for verifying bone resection during a surgical procedure, the method comprising:
coupling a plate to a generally longitudinal axis of a patient's bone;
coupling a post to the plate such that the post extends beyond the surgical site;
coupling a tracker component for navigation to the plate or to the post;
positioning the tracker component within the surgical site while remaining away from an articular surface resection area of the bone as a function of the post; and
verifying the bone resection while the tracker component remains within the surgical site during the resection of the bone.

20. The method of claim 19, wherein the surgical procedure is a knee replacement, and further comprising fixing the plate to the metaphyseal area of the tibia or the medial side of the femur.

21. The method of claim 19, wherein the bone is a femur and further comprising:
coupling a second plate having at least one hole to a flat surface of the patient's tibia;
coupling a second post coupled to the second plate such that the second post extends beyond the surgical site;
coupling a second tracker component for navigation to a generally longitudinal axis of the tibia;
positioning the second tracker component within the surgical site and away from the articular surface resection area of the tibia as a function of the second post; and
verifying the tibia resection while the second tracker component remains within the surgical site during the resection of the tibia.

22. The method of claim 21, further comprising fixing the plate on the medial side of the femur over ten millimeters above the distal end of the femoral articular surface and approximately midway between the anterior and posterior margins of the femoral surface.

23. The method of claim 21, further comprising coupling the second tracker component to both the second plate and the second post.

24. The method of claim 21, further comprising fixing the second plate at approximately ten millimeters below the proximal articular surface of the tibia and above the distal end of the tibial tubercle on the medial side of the tibia.

25. The method of claim 19, wherein the plate is triangular shaped, square shaped, hexagonal shaped or rectangular shaped.

26. The method of claim 19, wherein the plate is coupled to the bone by at least one locking screw.

27. The method of claim 19, further comprising coupling at least one support to the post that is operable to support a cutting jig for the bone resection.

28. The method of claim 19, further comprising coupling at least one blade to the post and operable for medial tissue retraction at the surgical site.

29. The method of claim 19, further comprising coupling the tracker component to both the plate and the post.

30. A method for verifying knee kinematics during a surgical procedure, the method comprising:
coupling a plate to a generally longitudinal axis of a patient's bone;
coupling a post to the plate such that the post extends beyond the surgical site;
coupling a tracker component for navigation to the plate or to the post;
positioning the tracker component within the surgical site while remaining away from an articular surface resection area of the bone as a function of the post; and
verifying the knee kinematics while the tracker component remains within the surgical site during the resection of the bone.

31. The method of claim 30, further comprising coupling at least one support to the post that is operable to support a cutting jig for the bone resection.

32. The method of claim 30, further comprising coupling at least one blade to the post and operable for medial tissue retraction at the surgical site.

33. The method of claim 30, further comprising coupling the tracker component to both the plate and the post.
